(19) United States
(12) Patent Application Publication
(10) Pub. No.: US 2016/0220326 A1
(43) Pub. Date: Aug. 4, 2016

(54) INVESTIGATION AND CONTROL DEVICE OF DRIVE SYSTEM

(71) Applicant: EPED INC., Kaohsiung City (TW)

(72) Inventors: TA-KO HUANG, KAOHSIUNG CITY (TW); ZONG-HAN LYU, KAOHSIUNG CITY (TW)

(21) Appl. No.: 14/828,064

(22) Filed: Aug. 17, 2015

(30) Foreign Application Priority Data
Feb. 4, 2015 (TW) 104103710

(51) Int. Cl. A61C 1/00; A61B 34/20

(52) U.S. Cl. CPC A61C 1/0015; A61B 34/20

(57) ABSTRACT

An investigation and control device of a drive system is provided, which controls a power source of a surgical instrument by using a drive investigation and control device, where the drive investigation and control device receives a signal of the guide positioning device and controls, according to a tracked connection state of the guide positioning device and the surgical instrument, whether the surgical instrument is driven; and a control panel on the drive investigation and control device electrically connects an instrumentation switch sensing device and at least one practice model sensing device, so that the drive investigation and control device controls, according to whether the instrumentation switch or/and the practice model is/are positioned correctly, whether the surgical instrument is normally driven, thereby completely and correctly controlling a correspondence between the surgical instrument and peripheral devices.

### CROSS-REFERENCE TO RELATED APPLICATION

This application claims the benefit of Taiwan Patent Application No. 104103710, filed on Feb. 4, 2015, which is hereby incorporated by reference for all purposes as if fully set forth herein.

### BACKGROUND

1. Technical Field

The present invention relates to an investigation and control device of a drive system, and particularly to an investigation and control device that can make guidance and operation records of a guide positioning device consistent with an actual situation through investigation and control by means of drive control.

2. Related Art

Current technologies for surgeries such as bone surgeries, abdominal surgeries and dental surgeries are interactive so that positions of medical devices are tracked. An aided-navigation dental treatment system uses a computer-aided navigation or tracing system to record a patient's teeth or jaw area by using a volume data set through, and positions of medical instruments and devices relative to the computer-aided navigation or tracing system are recorded.

In most dental guide positioning system technologies, a three-dimensional computed tomography (CT) image is used for photographing; a digital model of an area where a tooth is to be implanted is reconstructed; an optical tracking system is imported, and a position of a hand tool is tracked in an optical tracking manner; the image is processed in conjunction with use of the space positioning technology, so as to track an actual dental treatment area; coordinates of the two are connected; and the position of the hand tool is displayed in the CT image in real time, so that a depth and an angle of the hand tool is displayed in real time to a user during a dental training or dental surgery, and the user is provided with real-time data to determine whether the tool is out of alignment.

A dental teaching simulation machine or treatment table equipped with the action tracking and detection, positioning and guiding system can implement a three-dimensional digital record of a process of a surgery or practice operation. A surgical process including tooth turning, tooth implantation or injection requires accurate surgical operation behaviors, and the digital record can be used for guidance and indication during a surgery or operation recording and skill evaluation after a surgery. However, such a tracking and detection and positioning system has the following problems: due to limitations of the system, for example, the positioning system is an optical system, if there is a block between a tracking device and a hand tool, the whole system cannot perform detection and obtain a current related operation record, so that the operation process cannot be 100% consistent with the record. Therefore, guidance, operation recording and evaluation for a surgery or practice cannot be totally consistent with an actual situation.

### SUMMARY

An objective of the present invention is to provide an investigation and control device of a drive system, where a surgical instrument can be normally driven only when a guide positioning system, an instrumentation switch sensing device or/and a practice model sensing device are put in right actuation positions through the investigation and control device.

To solve the problems in the prior art, the present invention relates to an investigation and control device of a drive system, used in a dental treatment machine with a guide positioning device, where the dental treatment machine is provided with a surgical instrument, used for clinical or practice operations, and the surgical instrument is provided with a positioning element corresponding to the guide positioning device, characterized by including: a drive investigation and control device electrically connected between the dental treatment machine and the surgical instrument, where the drive investigation and control device receives a signal of the guide positioning device and controls, according to a tracked connection state of the guide positioning device and the surgical instrument, whether the surgical instrument is normally driven; and a control panel electrically connected to the drive investigation and control device, where the control panel electrically connects an instrumentation switch sensing device and at least one practice model sensing device, which are used for controlling an instrumentation switch and a practice model through investigation, so that the drive investigation and control device controls, according to whether the instrumentation switch or/and the practice model is/are positioned correctly, whether the surgical instrument can be normally driven.

The drive investigation and control device may be further disposed inside the dental treatment machine, and the drive investigation and control device is electrically connected with the guide positioning device and controls, according to the tracked connection state of the guide positioning device and the surgical instrument, whether the surgical instrument is normally driven by the dental treatment machine.

The guide positioning device has action tracking and detection, positioning and guiding functions, and when the guide positioning device can track and detect the surgical instrument, the drive investigation and control device enables the dental treatment machine to normally drive the surgical instrument; and when the guide positioning device cannot track the surgical instrument and detects that the surgical instrument is abnormally disconnected, the drive investigation and control device cuts off a power source through which the dental treatment machine drives the surgical instrument.

When the power source through which the surgical instrument is driven is fluid-driven, the drive investigation and control device controls circulation and blocking of a fluid flowing into a pipeline of the surgical instrument.

When the power source through which the surgical instrument is driven is electric-driven, the drive investigation and control device controls connection and disconnection of the power source to the surgical instrument.

The surgical instrument can be driven only when the control panel detects that the instrumentation switch equipped with the instrumentation switch sensing device is correctly turned on and positioned.

The surgical instrument can be normally driven only when the control panel detects that the practice model sensing device is correctly installed and positioned.

The present application has the following advantages: the drive investigation and control device receives a signal of the guide positioning device, so that the surgical instrument can be normally driven only when the guide positioning system cannot perform tracking and positioning due to a block or signal interference between the tracking device and the surgical instrument, when the guide positioning device cannot track the surgical instrument and detects that the surgical instrument is abnormally disconnected, and when the control panel detects that the instrumentation switch is correctly turned on and positioned, or after it is detected in practice that the practice model is correctly installed and positioned. Some instruments of the system are stopped by temporarily cutting off the driving power source of the surgical instrument through investigation and control, and the driving power source of the surgical instrument is restored after the guide positioning device is capable of tracking and detection and the peripheral devices have been disposed at proper positions, thereby completely and correctly controlling a correspondence between the surgical instrument and the peripheral devices. The surgical instrument can be normally driven only at right moment, so that digital recorded data is made consistent with an actual operation through investigation and control.

### BRIEF DESCRIPTION OF THE DRAWINGS

FIG. 1 is a schematic system view of an embodiment of the present application; and

FIG. 2 is a schematic system view of another embodiment of the present application.

### DETAILED DESCRIPTION

Detailed contents and technical description about the present invention are further described below with embodiments, but it should be understood that the embodiments are merely for illustration and should not be construed as limitations on implementation of the present invention.

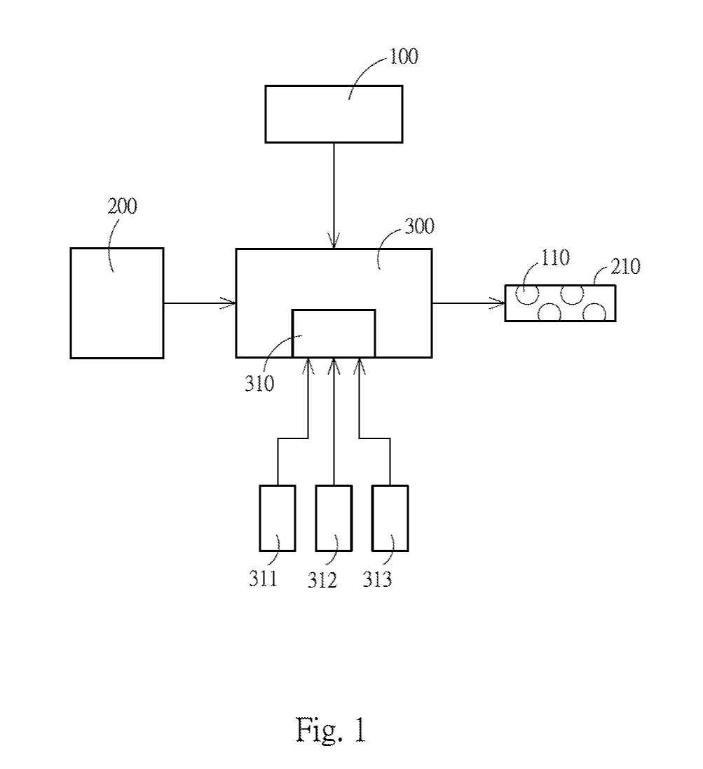

Referring to FIG. 1, FIG. 1 is a schematic system view of an embodiment of the present application. The present application relates to an investigation and control device of a drive system, used in a dental treatment machine 200 with a guide positioning device 100. The guide positioning device 100 has action tracking and detection, positioning and guiding functions, and can implement a three-dimensional digital record of a process of a surgery or practice operation. The dental treatment machine is provided with a surgical instrument 210, used for clinical or practice operations, and the surgical instrument 210 is provided with a positioning element 110 corresponding to the guide positioning device 100. Tracking and positioning are performed in an optical manner, an image is processed in conjunction with the space positioning technology, and a patient's oral and/or practice head model is positioned by using a tracking tool, so as to provide guidance and indication during a surgery or operation recording and skill evaluation after a surgery so that a mistake during usage can be corrected later. The guide positioning device 100 is a known prior art in the medical guide positioning system, which is not repeated herein.

Provided is a dental treatment machine 200 and a surgical instrument 210, extending from the dental treatment machine 200, to be used in a patient's oral and/or practice head model (not shown in the figure) for oral surgery or practice operations, for example, a dental handpiece, an electric-driven syringe, a tooth implantation machine and a tooth cleaning machine. The surgical instrument 210 held in hand by a physician or an intern is provided with the positioning element 110 corresponding to the guide positioning device 100, so that the guide positioning device 100 can track, detect and position actions of the surgical instrument 210.

A drive investigation and control device 300 is electrically connected between the dental treatment machine 200 and the surgical instrument 210, where the drive investigation and control device 300 receives a signal sent from the guide positioning device 100; in implementation applications, the signal may be received by using a wire or an existing known wireless technology, such as Bluetooth and wifi.

The drive investigation and control device 300 controls, according to a tracked connection state of the guide positioning device 100 and the surgical instrument 210, whether the surgical instrument 210 is normally driven. When the guide positioning device 100 can track and detect the action of the surgical instrument 210 and the guide positioning device 100 and the surgical instrument 210 are normally connected, that is, no block or signal interference occurs between the tracking device of the guide positioning device 100 and the surgical instrument 210 to affect tracking and positioning, the drive investigation and control device 300 enables the dental treatment machine 200 to normally drive the surgical instrument 210. When the guide positioning device 100 cannot track and detect the surgical instrument 210 and the guide positioning device 100 and the surgical instrument 210 are abnormally disconnected, that is, a block occurs between the tracking device of the guide positioning device 100 and the surgical instrument 210, the block may be in the device or it may be that a user (physician or intern) blocks a connecting path, the drive investigation and control device 300 cuts off a power source through which the surgical instrument 210 is driven.

Also, a control panel 310 is electrically connected to the drive investigation and control device 300, where the control panel 310 is used for electrically connecting an instrumentation switch sensing device 311 and at least one practice model sensing device 312, 313. The practice model sensing devices 312, 313 may be sensing devices of dental practice peripheral models such as maxillary and mandibular models for practice, dental models for tooth implantation practice, prosthesis practice single tooth, and injection site models. In implementation, the drive investigation and control device 300 controls, according to whether the instrumentation switch sensing device 311 or/and the practice model sensing devices 312, 313 (in FIG. 1, two sensing devices are taken as an example; in implementation, the number of sensing devices connected to a port of the control panel 310 may be increased or decreased according to a requirement on the number of connected sensing devices) is/are correctly positioned, whether the surgical instrument 210 is normally driven.

The instrumentation switch sensing device 311 is disposed at an instrumentation switch, such as a pedal switch or a shift switch used in the dental handpiece, while an electric pressing switch used in a syringe is controlled and started by a user (physician or intern), and the surgical instrument 210 can be driven after the user (physician or intern) completes preparation and correctly starts the instrumentation switch, which prevents unnecessary waste of power sources and prevents non-user-induced incorrect driving.

When a practice head model is used, the control panel 310 detects whether the practice model sensing devices 312, 313 are correctly installed, for example, only when a maxillary sensing device and a mandibular sensing device are correctly installed and positioned in the practice head model, the surgical instrument 210 can be normally driven. In implementation, the sensing devices may be reed sensing switches, Hall effectors, press (short circuit) switches, light interrupt switches or other sensing switches that have been technically mature in the market and have installation sensing functions. For example, a reed sensing switch is added to the maxillary sensing device and the mandibular sensing device, to detect, through magnetic attraction and sensing of the head model, whether the maxillary sensing device and the mandibular sensing device are separated from the practice head model, and the practice environment of the intern, and whether the devices in the practice head model are correctly arranged, so that the intern can practice in a correct practice situation. For example, turning can be performed only when the dental model is placed in the head model, and teeth cannot be turned without taking out the dental model.

When the power source through which the surgical instrument 210 is driven is fluid-driven, the drive investigation and control device 300 controls circulation and blocking of a fluid flowing into a pipeline of the surgical instrument 210. For example, the dental handpiece uses a pressure gas as a turning torque source, and in implementation, the drive investigation and control device 300 can block a pipeline between the dental treatment machine 200 and the surgical instrument 210 in a known mechanical or electromagnetic technical manner, to temporarily stop supplying a fluid that drives the surgical instrument 210.

When the power source through which the surgical instrument 210 is driven is electric-driven, the drive investigation and control device 300 controls connection and disconnection of the power source to the surgical instrument 210. For example, the surgical instrument 210 is a motor, an electric-driven syringe, or another electronic-controlled surgical instrument, and in implementation, the drive investigation and control device 300 can completely or partially disconnect the power supply between the dental treatment machine 200 and the surgical instrument 210 in an electronic control manner, to temporarily stop supplying power that drives the surgical instrument 210.

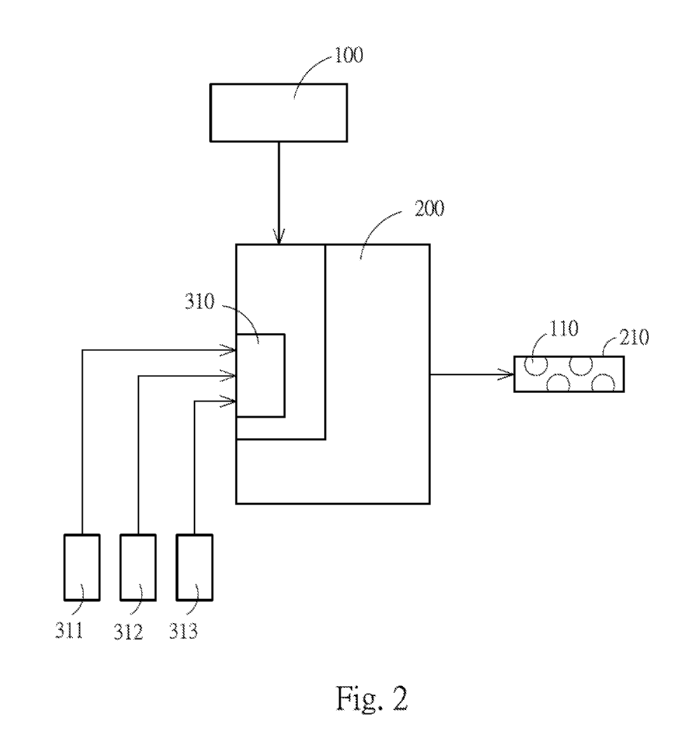

Referring to FIG. 2, in implementation of the present invention, the drive investigation and control device 300 may also be directly disposed in the dental treatment machine 200, and control, according to a tracked connection state of the guide positioning device 100 and the surgical instrument 210, whether the dental treatment machine 200 provides a driving power source to the surgical instrument 210. When the guide positioning device 100 can track the surgical instrument 210 and detects that the surgical instrument 210 is normally connected, the dental treatment machine 200 drives the surgical instrument according to a normal operation state. However, when the guide positioning device 100 cannot track the surgical instrument and detects that the surgical instrument is abnormally disconnected 210, the drive investigation and control device 300 cuts off the power source of the dental treatment machine 200 to the surgical instrument 210, so that the dental treatment machine 200 loses part of the power source and cannot normally drive the surgical instrument 210.

In the same way, the instrumentation switch of the instrumentation switch sensing device 311 is controlled and started by a user (physician or intern), and the dental treatment machine 200 can normally drive the surgical instrument 210 only after the user (physician or intern) completes preparation and correctly starts the instrumentation switch.

As stated above, only when a practice head model is used and it is detected through the control panel 310 that the practice model sensing devices 312, 313 are correctly installed in the practice device, the dental treatment machine 200 can normally drive the surgical instrument 210.

When the power source through which the surgical instrument 210 is driven is fluid-driven, the drive investigation and control device 300 controls circulation and blocking of a fluid in the dental treatment machine 200. For example, the dental handpiece uses a pressure gas as a turning torque source, and in implementation, the drive investigation and control device 300 can block a pipeline through which gas flows into the whole dental treatment machine 200 in a known mechanical or electromagnetic technical manner, so that the dental treatment machine 200 cannot supply a fluid that drives the surgical instrument 210.

When the power source through which the surgical instrument 210 is driven is electric-driven, the drive investigation and control device 300 controls connection and disconnection of the power source to the dental treatment machine 20. For example, a motor, an electric-driven syringe or another electronic-controlled surgical instrument may completely disconnect the power supply of the dental treatment machine 200 in an electronic control manner, so that the dental treatment machine 200 cannot supply power that drives the surgical instrument 210.

It is worth mentioning that the selection between complete and partial disconnection of the power supply in implementation should be determined according to the state of the surgical instrument 210. Using a tooth implantation machine as an example, if the power supply is completely disconnected and then connected, the entire system cannot be quickly restarted for use; therefore, in implementation, it is feasible to install the drive investigation and control device 300 on a motor line of the surgical instrument 210 for partial disconnection.

However, the above descriptions are merely preferred embodiments of the present invention, and cannot be used to limit the scope of implementation of the present invention. Any simple and equivalent variations and modifications made according to CLAIMS and SUMMARY of the present invention should fall within the scope of the present invention.

What is claimed is:

1. An investigation and control device of a drive system, used in a dental treatment machine with a guide positioning device, wherein the dental treatment machine is provided with a surgical instrument, used for clinical or practice operations, and the surgical instrument is provided with a positioning element corresponding to the guide positioning device, characterized by comprising:
a drive investigation and control device electrically connected between the dental treatment machine and the surgical instrument, wherein the drive investigation and control device receives a signal of the guide positioning device and controls, according to whether the guide positioning device and the surgical instrument are in a trackable connection state, whether the surgical instrument can be driven; and
a control panel electrically connected to the drive investigation and control device, wherein the control panel electrically connects an instrumentation switch sensing device and at least one practice model sensing device, which are used for controlling an instrumentation switch and a practice model through investigation, so that the drive investigation and control device controls, according to whether the instrumentation switch or/and the practice model is/are positioned correctly, whether the surgical instrument can be normally driven.

2. An investigation and control device of a drive system, used in a dental treatment machine with a guide positioning device, wherein the dental treatment machine is provided with a surgical instrument, used for clinical or practice operations, and the surgical instrument is provided with a positioning element corresponding to the guide positioning device, characterized by comprising:
a drive investigation and control device electrically connected to the inside of the dental treatment machine, wherein the drive investigation and control device receives a signal of the guide positioning device and controls, according to whether the guide positioning device and the surgical instrument are in a trackable connection state, whether the surgical instrument can be driven by the dental treatment machine; and
a control panel electrically connected to the drive investigation and control device, wherein the control panel electrically connects an instrumentation switch sensing device and at least one practice model sensing device, which are used for controlling an instrumentation switch and a practice model through investigation, so that the drive investigation and control device controls, according to whether the instrumentation switch or/and the practice model is/are positioned correctly, whether the surgical instrument can be normally driven by the dental treatment machine.

3. The investigation and control device of a drive system according to claim 1, wherein the guide positioning device has action tracking and detection, positioning and guiding functions, and when the guide positioning device can track and detect the surgical instrument, the drive investigation and control device enables the dental treatment machine to normally drive the surgical instrument; and when the guide positioning device cannot track the surgical instrument and detects that the surgical instrument is abnormally disconnected, the drive investigation and control device cuts off a power source through which the dental treatment machine drives the surgical instrument.

4. The investigation and control device of a drive system according to claim 2, wherein the guide positioning device has action tracking and detection, positioning and guiding functions, and when the guide positioning device can track and detect the surgical instrument, the drive investigation and control device enables the dental treatment machine to normally drive the surgical instrument; and when the guide positioning device cannot track the surgical instrument and detects that the surgical instrument is abnormally disconnected, the drive investigation and control device cuts off a power source through which the dental treatment machine drives the surgical instrument.

5. The investigation and control device of a drive system according to claim 3, wherein, when the power source through which the surgical instrument is driven is fluid-driven, the drive investigation and control device controls circulation and blocking of a fluid flowing into a pipeline of the surgical instrument.

6. The investigation and control device of a drive system according to claim 3, wherein, when the power source through which the surgical instrument is driven is electric-driven, the drive investigation and control device controls connection and disconnection of the power supply to the surgical instrument.

7. The investigation and control device of a drive system according to claim 3, wherein the surgical instrument can be driven only when the control panel detects that the instrumentation switch equipped with the instrumentation switch sensing device is correctly turned on and positioned.

8. The investigation and control device of a drive system according to claim 3, wherein the surgical instrument can be normally driven only when the control panel detects that the practice model equipped with the practice model sensing device is correctly installed and positioned.
